(19) United States
(12) Patent Application Publication
(10) Pub. No.: US 2026/0108553 A1
(43) Pub. Date: Apr. 23, 2026

(54) MAGNESIUM SALTS COMPOSITION

(71) Applicant: Instituto de Dermatología Ignacio Umbert, S.L., Barcelona (ES)

(72) Inventor: Ignacio UMBERT MILLET, Barcelona (ES)

(73) Assignee: Instituto de Dermatología Ignacio Umbert, S.L., Barcelona (ES)

(21) Appl. No.: 18/997,843

(22) PCT Filed: Jul. 17, 2024
(86) PCT No.: PCT/ES2024/070458

§ 371 Date: Jan. 23, 2025

(30) Foreign Application Priority Data
Jul. 25, 2023 (EP) 23382773.2

(51) Int. Cl. A61K 33/06; A61K 33/14; A61K 33/16; A61K 36/16; A61K 36/23; A61K 36/258; A61K 36/28; A61K 36/38; A61K 36/48; A61K 36/537; A61K 36/77; A61K 36/82; A61K 36/899; A61K 45/06; A61P 17/00

(52) U.S. Cl. CPC A61K 33/06; A61K 33/14; A61K 33/16; A61K 36/16; A61K 36/23; A61K 36/258; A61K 36/28; A61K 36/38; A61K 36/48; A61K 36/537; A61K 36/77; A61K 36/82; A61K 36/899; A61K 45/06; A61P 17/00

(57) ABSTRACT

The present invention relates to a composition, characterised in that it comprises at least one magnesium salt, at least one salt not comprising magnesium, at least one antioxidant, and at least one plant extract. The composition of the invention is intended for the treatment of pathologies of inflammation of the skin.

### TECHNICAL FIELD OF THE INVENTION

The present invention relates to a composition, characterised in that it comprises at least one magnesium salt, at least one salt not comprising magnesium, at least one antioxidant, and at least one plant extract. The composition of the invention is intended for the treatment of inflammation, in particular inflammation in the skin, for example: dermatitis, atopic dermatitis, vitiligo, psoriasis or pruritus, and combinations thereof.

### BACKGROUND OF THE INVENTION

Skin inflammation is a problem that can be caused by various factors, such as environmental factors, allergies, irritants, medical conditions, stress, and genetics. It is a common condition characterised by redness, itching, swelling, and a burning sensation on the surface of the skin In addition, it is essential to take care of the skin and take measures to prevent autoimmune lesions at a cutaneous and systemic level (such as psoriasis, atopic dermatitis, itching, vitiligo and cancer prevention) and other processes.

Existing technology to treat inflammation in the skin includes creams, lotions, or ointments containing topical corticosteroids, antihistamines, and other active ingredients. However, these treatments often have unwanted side effects and are not effective for everyone. Some medications, such as certain antibiotics, non-steroidal anti-inflammatory drugs, and hypertension medications, can also trigger or aggravate the disease in certain individuals.

In addition, the use of natural ingredients in the formulation of products to treat skin inflammation has been investigated. For example, aloe vera and calendula have been shown to have anti-inflammatory and soothing properties that can help reduce skin inflammation. However, these natural ingredients are often unstable and may lose their effectiveness in the manufacturing and/or application process.

In short, adequate prevention of skin inflammation at a systemic or local level and skin protection are crucial measures to avoid neoplastic skin and oral lesions in addition to preventing autoimmune disorders in the body. Accordingly, there is an unmet need for an effective and safe composition for treating irritation that does not have unwanted side effects and that contains stable and effective natural ingredients.

In the prior art, several compositions containing magnesium are known. For example, document US2008/038219 A1 discloses a topical composition comprising carboxylic acids, chelating agents, dimethyl sulfone, and magnesium sulphate, which serves as a base for the application of drugs and active ingredients. Additionally, document WO 2020/095099 discloses compositions that include magnesium, as well as various salts and plant extracts. However, these compositions are intended for oral cosmetic use due to their remineralizing activity, which restores dental enamel and reduces tooth sensitivity.

Similarly, there are skincare products that contain magnesium salts, non-magnesium salts, plant extracts, and antioxidants, such as facial masks. However, these products are not intended for the treatment of skin inflammation and/or irritation but rather for daily skincare.

The present invention meets this need by the formulation of an innovative composition utilising safe, natural active ingredients that have been shown to be effective in treating inflammation, both that occurring in the skin and that occurring in the mouth.

### SUMMARY OF THE INVENTION

The present invention resolves the aforementioned drawbacks by means of a composition comprising at least one magnesium salt among other natural ingredients.

The first aspect of the invention relates to a composition characterised in that it comprises at least one magnesium salt, at least one salt not comprising magnesium, at least one antioxidant, and at least one plant extract.

The combination of compounds of the composition results in an increase in anti-inflammatory efficacy. Thus, in general, the resulting formulations of the present invention allow eliminating from the composition the aggressive substances such as corticosteroids, antibiotics, enhancing, surprisingly, by a synergistic effect, the anti-inflammatory effect of the compounds of the composition at low concentrations.

The second aspect of the invention relates to the process of preparing the composition according to the first aspect.

The third aspect of the invention relates to the use of the composition of the first aspect for the treatment of inflammation in the skin, for example, in the treatment of dermatitis, atopic dermatitis, vitiligo, psoriasis or pruritus.

### BRIEF DESCRIPTION OF THE DRAWINGS

FIG. 1 shows the evolution in the affected skin area before (1A, 1C) and after (1B, 1D) treatment with the composition object of the invention.

FIG. 2 shows a comparison of SCORAD, DLQI, EASI, POEM, PRURITO, and VLG-AD values measured at two different times: first before the treatment and then 15 days after applying the composition. These values are compared between the compositions of the invention and those known in the prior art.

### DETAILED DESCRIPTION OF THE INVENTION

The first aspect of the invention relates to a composition characterised in that it comprises at least one magnesium salt, at least one salt not comprising magnesium, at least one antioxidant, and at least one plant extract. Preferably, the composition of the invention is an aqueous composition, although it may also be formulated as an alcoholic composition. The term “aqueous composition” refers to a composition containing water as the main solvent. In particular, the compositions of the invention comprise at least 95% by weight of water, preferably at least 98% by weight of water in relation to the composition, more preferably 99% by weight of water in relation to the composition. The term “alcoholic composition” relates to a composition containing an alcohol as a solvent, e.g., ethanol. In particular, the alcoholic compositions of the invention comprise at least 10% by weight of alcohol. Unless otherwise indicated, all percentages refer to percentages by weight with respect to the composition.

In a preferred embodiment of the first aspect of the invention, the at least one magnesium salt is selected from organic, inorganic magnesium salts or mixtures thereof. The inorganic magnesium salts are selected from those salts having a high solubility in water, such as halides or sulphates. It is preferably selected from the group of magnesium sulphate, magnesium chloride, magnesium bromide, magnesium oxide or mixtures thereof. In a preferred embodiment the magnesium salt is magnesium sulphate. In another preferred embodiment, the magnesium salt is a mixture of magnesium sulphate and magnesium chloride. The organic magnesium salts are selected from those salts having a high solubility in water, such as, for example, citrates, aspartates or lactates.

In a preferred embodiment of the first aspect of the invention, the composition comprises between 0.01 and 3% by weight with respect to the composition of at least one salt comprising magnesium. Preferably between 0.01 and 2% by weight with respect to the composition.

In a preferred embodiment of the first aspect of the invention, the at least one salt not comprising magnesium is selected from the group of inorganic salts with high solubility comprising halogenides, borates, sulphates, carbonates and bicarbonates in combination with sodium, calcium, potassium, chlorine and sulphur. In particular the salts are selected from the group of sodium chloride, sodium fluoride, potassium chloride, sodium sulphate, calcium chloride, sodium bicarbonate, sodium bromide, strontium chloride, sodium borate, ferric chloride or mixtures thereof. In a preferred embodiment, the non-magnesium comprising salt is a mixture of sodium chloride, sodium fluoride and potassium chloride. In another preferred embodiment, the salt that does not contain magnesium is a mixture of sodium chloride, sodium fluoride, potassium chloride, and ferric chloride.

In a preferred embodiment of the first aspect of the invention, the composition comprises between 0.01 and 3% by weight with respect to the composition of at least one salt not comprising magnesium. Preferably between 0.01 and 2.5% by weight with respect to the composition.

In a preferred embodiment of the first aspect of the invention, the composition comprises at least one plant extract selected from solid plant extracts or glycolic plant extracts, or a mixture thereof. The term “glycolic plant extract” refers to the form of botanical extract in which propylene glycol-based solvents are used to obtain the active compounds from plants, that is a solution of propylene glycol that contains the plant extract.

In a preferred embodiment of the first aspect of the invention, the composition comprises between 0.01 and 0.1% by weight in relation to the composition of at least one plant extract. Preferably between 0.01 and 0.05% by weight in relation to the composition.

In a preferred embodiment, the solid plant extract is selected from the group of rosemary dry extract, fermented papaya extract, green tea extract, thyme dry extract, chamomile dry extract, lemon balm dry extract, witch hazel dry extract, ginseng dry extract, Ginkgo biloba dry extract and hypericum dry extract, curcuma dry extract or mixtures thereof. The preferred solid plant extract is a mixture of rosemary dry extract, fermented papaya extract, green tea extract. The term “dry extract” refers to a plant extract in powder or solid form.

In a preferred embodiment, the glycolic plant extract is selected from the group of chamomile glycolic extract, oat glycolic extract, sage glycolic extract, Centella asiatica glycolic extract, birch glycolic extract, flowering calendula glycolic extract, melilot glycolic extract, white nettle glycolic extract, green nettle glycolic extract and horse chestnut glycolic extract or mixtures thereof. The preferred glycolic plant extract is a mixture of chamomile glycolic extract, glycolic oat extract and glycolic sage extract.

In an even more preferred embodiment, the composition comprises rosemary dry extract, fermented papaya extract, green tea extract, chamomile glycolic extract, glycolic oat extract and glycolic sage extract.

In a preferred embodiment of the first aspect of the invention, the composition comprises at least one antioxidant selected from the group of ferulic acid, nicotinamide, zinc gluconate, iron gluconate, melatonin, resveratrol, vitamin C (ascorbic acid), dimethylethanolamine (DMAE), glycolic acid and coenzyme Q, lipoic acid, quercetin, curcumin, ascorbic palmitate, taurine, isoflavones, lycopene, vitamin E, L-carnosine, or mixtures thereof. Preferably, the at least one antioxidant is a mixture of ferulic acid, nicotinamide, zinc gluconate, melatonin, and resveratrol.

Ferulic acid is a natural antioxidant found in various plants, especially in grains such as rice, wheat and oats, as well as in some fruits and vegetables. It is known for its antioxidant and anti-inflammatory properties, making it a valuable component in skin care products and dietary supplements. Helps protect the skin from damage caused by free radicals and contributes to the prevention of premature ageing of the skin

Nicotinamide, also known as vitamin B3 or niacinamide, is a form of water-soluble vitamin B. It plays an essential role in cellular metabolism and has anti-inflammatory properties and the ability to help improve skin tone, reduce the appearance of dark spots and minimise enlarged pores. In addition, it contributes to maintaining the protective barrier of the skin and adequate hydration.

Zinc gluconate is a zinc salt that is frequently used as a nutritional supplement and is also found in skin care products. Zinc is an essential trace element for the functioning of the body, including the maintenance of the immune system and cell regeneration. In skin care, zinc gluconate can help regulate sebum production, reduce inflammation, and promote wound healing.

Melatonin, also known as N-acetyl-5-methoxytryptamine, is a hormone found in higher animals and some algae. In the skin, melatonin plays a crucial role as a free radical scavenger. It increases the activity of enzymes responsible for metabolising free radicals and, in addition, is able to oxidise to eliminate them without producing a redox cycle, which has led it to be called a “suicide antioxidant”.

Resveratrol is a polyphenolic compound found in some plants, especially in the skin of red grapes, and also in red wine. It has antioxidant and anti-inflammatory properties that may be beneficial for cardiovascular health and skin. It has been studied for its possible effects in reducing cellular ageing and in protecting against damage caused by free radicals.

By combining the different antioxidants, the synergistic effect of the composition is enhanced, thus achieving a greater benefit for the skin or the body's antioxidant system.

In a preferred embodiment of the first aspect of the invention, the composition comprises between 0.01 and 1% by weight relative to the aqueous composition of at least one antioxidant. Preferably between 0.01% and 0.1% by weight relative to the composition of at least one antioxidant. More preferably, between 0.01% and 0.05% by weight relative to the composition.

In the most preferred embodiment the composition comprises:
0.1 and 1% by weight in relation to the magnesium sulphate composition;
0.1 and 1% by weight in relation to the composition of sodium chloride, sodium fluoride and potassium chloride;
0.01 and 0.1% by weight in relation to the composition of rosemary dry extract, fermented papaya extract, green tea extract and chamomile glycolic extract; and
0.01 and 0.1% by weight in relation to the composition of ferulic acid, nicotinamide, zinc gluconate, melatonin, and resveratrol.

In another most preferred embodiment the composition comprises:
0.1 and 1% by weight in relation to the magnesium sulphate composition;
0.1 and 1% by weight in relation to the composition of sodium chloride, sodium fluoride and potassium chloride;
0.01 and 0.1% by weight in relation to the composition of rosemary dry extract, fermented papaya extract, green tea extract and chamomile glycolic extract; and
0.01 and 0.1% by weight in relation to the composition of ferulic acid, nicotinamide, zinc gluconate, melatonin, and resveratrol.

In another most preferred embodiment the composition comprises:
0.1 and 3% by weight in relation to the magnesium sulphate and magnesium chloride composition;
0.1 and 3% by weight in relation to the composition of sodium chloride, sodium fluoride and potassium chloride;
0.01 and 0.1% by weight in relation to the composition of rosemary dry extract, fermented papaya extract, green tea extract, chamomile glycolic extract, glycolic oat extract and glycolic sage extract;
0.01 and 0.1% by weight in relation to the composition of ferulic acid, nicotinamide, zinc gluconate, melatonin, and resveratrol.

In another most preferred embodiment the composition comprises:
0.1 and 3% by weight in relation to the magnesium sulphate and magnesium chloride composition;
0.1 and 3% by weight in relation to the composition of sodium chloride, sodium fluoride and potassium chloride;
0.01 and 1% by weight in relation to the composition of rosemary dry extract, fermented papaya extract, green tea extract, chamomile glycolic extract, glycolic oat extract and glycolic sage extract;
0.01 and 1% by weight in relation to the composition of ferulic acid, nicotinamide, zinc gluconate, melatonin, and resveratrol.

The compositions of the examples can be completed with variable amounts of other substances, such as, for example, preservatives, pigments, thickeners, aromatic agents or active ingredients, according to the intended use of the composition.

In other preferred embodiments of the composition, it additionally comprises at least one element selected from rosehip oil, jojoba oil, calendula oil, shea butter, medium-chain triglycerides (fatty acid esters with chain lengths between 8 and 12 carbon atoms and glycerol (1,2,3-propanetriol)), dimethicone, glycerin, dexpanthenol, urea, hyaluronic acid, enoxolone, lanolin, or mixtures thereof. The inclusion of one or more of these components enhances the anti-inflammatory, antiviral, and antimicrobial properties of the composition. For example, jojoba oil or medium-chain triglycerides provide these qualities, while dimethicone or glycerin improve its moisturizing and healing capabilities, among others.

In other preferred embodiments of the composition, it is in any pharmaceutically acceptable form for topical application, for example, in the form of aqueous compositions, alcoholic compositions, gel, cream, cream-gel or lotion.

The second aspect of the invention relates to a process of preparing the composition of the first aspect of the invention.

In the preferred embodiment of the second aspect, the method of preparing a composition according to the first aspect comprises the following steps:
a) Providing the desired amount of solvent;
b) Adding at least one magnesium salt and at least one salt not comprising magnesium to the solvent from the previous step;
c) Adding at least one antioxidant and at least one plant extract to the mixture of the previous stage.
d) Optionally, adding excipients such as preservatives, antioxidants, pigments, thickeners, aromatic agents or active ingredients, according to the intended use of the composition.

The third aspect of the invention relates to the use of the composition of the first aspect for the treatment of inflammation.

In an embodiment of the third aspect of the invention, the use of the composition according to the first aspect of the invention is a therapeutic use for the treatment and/or prevention of inflammation in the skin, for example, in the treatment of dermatitis, atopic dermatitis, vitiligo, psoriasis or pruritus.

In a preferred embodiment of the third aspect of the invention, the treatment of the affected area of the skin is carried out by immersing the affected area in the composition of the first aspect for a certain period of time.

In another preferred embodiment of the third aspect of the invention, the treatment of the affected area of the skin is carried out by spraying the affected area with the composition of the first aspect.

In another preferred embodiment of the third aspect of the invention, the treatment of the affected area of the skin is carried out by applying the composition in gel, cream or cream-gel format of the first aspect to the affected area.

In each case and for each patient, the doctor will assess—with the appropriate periodicity—the possible presence and intensity of the pathologies, and will formulate the composition and its application depending on one or the other.

With the compositions of the present invention, inflammation is controlled without the need to abuse substances with side effects, such as antibiotics or corticosteroids, by surprisingly enhancing the effect of the components of the composition of the invention present at low concentrations.

### EXAMPLES

### Example 1: Preparation of the Aqueous Composition of the Invention

The preferred embodiment of the invention is detailed below, by way of non-limiting example

| | Concentration |
|---|---|
| Component | (wt %) |
| Magnesium sulphate | 0.31 |
| Sodium chloride | 0.17 |
| Sodium fluoride | 0.006 |
| Potassium chloride | 0.06 |
| Rosemary dry extract | 0.009 |
| Fermented papaya extract | 0.006 |
| Green tea extract | 0.0006 |
| Ferulic Acid | 0.0006 |
| Nicotinamide | 0.03 |
| Zinc gluconate | 0.009 |
| Melatonin | 0.0000006 |
| Resveratrol | 0.0000009 |
| Chamomile glycolic extract | 0.0000006 |
| Water | Q.S. |

The process for preparing the aqueous composition of the invention comprises the following steps:
a) Providing the necessary amount of water according to the area of the skin to be treated;
b) Adding magnesium sulphate, sodium chloride, sodium fluoride, potassium chloride, ferulic acid, nicotinamide, zinc gluconate, rosemary dry extract, fermented papaya extract, and water-based green tea extract from the previous step;
c) Adding melatonin, resveratrol, and chamomile glycolic extract to the dissolution from the previous stage.

As indicated, the amount of water may vary depending on the area to be treated: if total immersion is required, 100 L (a bathtub) will be prepared, if partial immersion is required, 50 L (a basin), 10 L (a bidet) or 5 L (a basin) of the composition of the invention will be prepared.

In addition to the application of this composition in bathtubs and/or showers, it can also be applied through a vaporiser spray.

### Example 2: Preparation of the Composition of the Invention—Gel, Cream or Gel-Cream

Other preferred embodiments of the invention are detailed below, by way of non-limiting example. In particular, example 2.1 corresponds to a preparation in cream-gel format, example 2.2 to a preparation in gel format and 2.3 to a composition in cream format

| | Example | Example | Example |
|---|---|---|---|
| Component | 2.1 (wt %) | 2.2 (wt %) | 2.3 (wt %) |
| NaCl | 0.336 | 0.336% | 0.336% |
| Ferulic Acid | 0.012 | 0.012 | 0.012 |
| NaF | 0.012 | 0.012 | 0.012 |
| KCl | 0.12 | 0.12 | 0.12 |
| Nicotinamide | 0.06 | 0.06 | 0.06 |
| MgSO₄ | 0.6216 | 0.6216 | 0.6216 |
| Papaya dry extract | 0.012 | 0.012 | 0.012 |
| Zn gluconate | 0.018 | 0.018 | 0.018 |
| Rosemary dry extract | 0.018 | 0.018 | 0.018 |
| Green tea dry extract | 0.0012 | 0.0012 | 0.0012 |
| Chamomile glycolic | 0.006 | 0.006 | 0.006 |
| extract | | | |
| Melatonin | 0.004 | 0.004 | 0.004 |
| Resveratrol | 0.004 | | |
| Water-alcohol solution | 0.186 | 0.186 | 0.186 |
| Vitamin C | 1 | — | 1 |
| Vitamin E-acetate | 11 | — | 1 |
| Aqua conservans | 20 | 20 | 20 |
| Cream-gel emulsion | Q.S. | — | — |
| Carboxymethylcellulose | | Q.S. | |
| [symbol] | | | |
| Oil-in-water emulsion | — | — | Q.S. |

[symbol]

indicates data missing or illegible when filed

### Example 3: Treatment of Skin Carried Out by Applying the Composition of the Invention

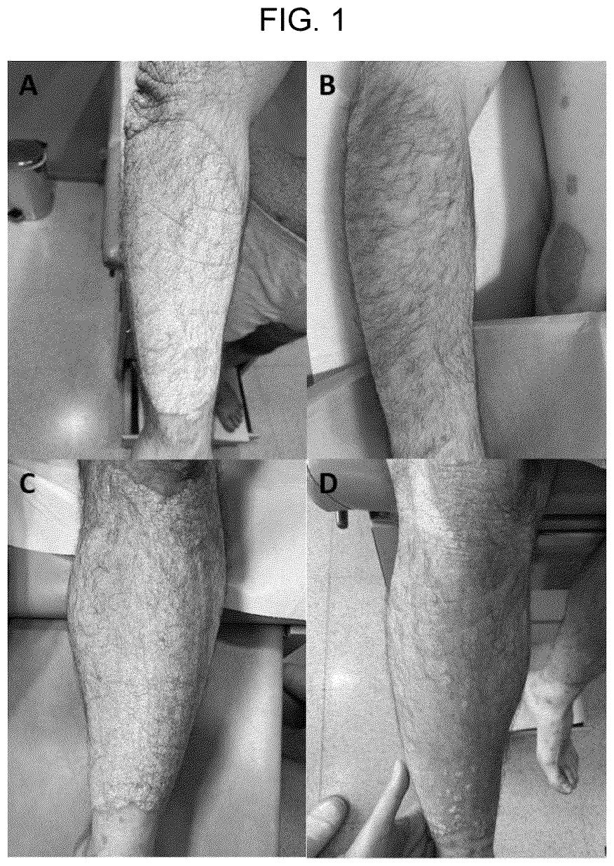

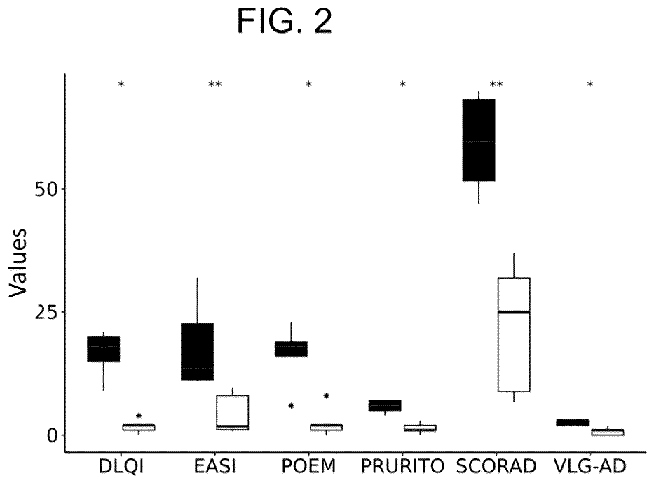

The composition of the invention, in particular that prepared according to example 1, is applied to patients by total immersion of the patient or the patient's area for at least 20 minutes. The results of the treatment can be seen in FIGS. 1 and 2 where the before (1A, 1C) and after (1B, 1D) treatment with the compositions of the invention can be observed. As can be seen in the figure, the decrease in inflammation is remarkable.

Alternatively, the compositions of the invention can be applied by spraying on the area to be treated. Other forms of the composition, examples 2.1-2.3, are formulated so that they can be applied directly to the desired area without the need to submerge the affected area. The results obtained are comparable to those obtained with the treatment of the composition of example 1.

### Example 4. Efficacy of the Compositions of the Invention

The efficacy of the compositions known in the prior art, see US2008/038219 A1 table 1, was compared against the compositions of the invention, with particular emphasis on example 2.

The compared parameters were as follows:
SCORAD (Scoring Atopic Dermatitis): SCORAD is a clinical tool used to assess the severity of atopic dermatitis (eczema). It considers the extent and intensity of the rash, as well as other symptoms such as itching and sleep disturbance.
DLQI (Dermatology Life Quality Index): DLQI is a questionnaire that measures the impact of skin conditions on a patient's quality of life.
EASI (Eczema Area and Severity Index): EASI is another tool for evaluating the severity of eczema. It assesses the extent and intensity of erythema (redness), induration (swelling), excoriation (scratching), and lichenification (thickening) in different body regions affected by eczema.
POEM (Patient-Oriented Eczema Measure): POEM is a patient-reported outcome measure used to assess the severity of eczema symptoms from the patient's perspective. It evaluates symptoms such as itchiness, sleep disturbance, bleeding, oozing, and impact on daily activities over the past week.
PRURITO (Pruritus Severity Scale): PRURITO is a scale used to assess the severity of pruritus (itching) experienced by patients with skin conditions. It evaluates the intensity and frequency of itching, as well as its impact on daily activities and quality of life.
VLG-AD (Visual Analog Scale for Atopic Dermatitis): VLG-AD is a visual analog scale used to assess the severity of atopic dermatitis. Patients mark their level of disease severity on a line ranging from “no symptoms” to “very severe symptoms.”

The study was conducted with 10 volunteers, distributed equally between the two creams. For each sample, values of SCORAD, DLQI, EASI, POEM, PRURITO, and VLG-AD were collected at two different time points: first before treatment and then again 15 days after the application of the cream.

As shown in FIG. 2, the compositions of the invention (European cream) exhibit a superior and enhanced effect on all measured metrics compared to the prior art compositions (American cream), with a particular emphasis on SCORAD. This clearly demonstrates the improved and unexpected effect of the compositions of the invention.

1.-14. (canceled)

15. A topical anti-inflammatory composition for use in a treatment of skin inflammation, characterized in that it comprises at least one magnesium salt, at least one salt not comprising magnesium, at least one antioxidant, and at least one plant extract, wherein the at least one salt not comprising magnesium is a mixture of sodium chloride, sodium fluoride, and potassium chloride.

16. The composition for use in a treatment of skin inflammation according to claim 15, wherein the at least one salt comprising magnesium is selected from the group of magnesium sulphate, magnesium chloride, magnesium bromide or mixtures thereof.

17. The composition for use in a treatment of skin inflammation according to claim 15, wherein the at least one salt comprising magnesium is magnesium sulphate.

18. The composition for use in a treatment of skin inflammation according to claim 15, wherein the at least one salt comprising magnesium is in a concentration of 0.1 to 1% by weight in relation to the composition.

19. The composition for use in a treatment of skin inflammation according to claim 15, wherein the at least one salt not comprising magnesium is in a concentration of 0.1 to 3% by weight relative to the composition.

20. The composition for use in a treatment of skin inflammation according to claim 15, wherein the at least one plant extract is a chamomile glycolic extract, oat glycolic extract, sage glycolic extract, Centella asiatica glycolic extract, birch glycolic extract, calendula glycolic extract, melilot glycolic extract, white nettle glycolic extract, green nettle glycolic extract, and horse chestnut glycolic extract, or mixtures thereof.

21. The composition for use in a treatment of skin inflammation according to claim 15, wherein the at least one plant extract is a solid extract selected from rosemary dry extract, fermented papaya extract, green tea extract, thyme dry extract, chamomile dry extract, lemon balm dry extract, witch hazel dry extract, ginseng dry extract, Ginkgo biloba dry extract, and hypericum dry extract, or mixtures thereof.

22. The composition for use in a treatment of skin inflammation according to claim 15, wherein the at least one plant extract is in a concentration of 0.01 to 0.1% by weight relative to the composition.

23. The composition for use in a treatment of skin inflammation according to claim 15, wherein the at least one antioxidant is selected from the group of ferulic acid, nicotinamide, zinc gluconate, melatonin, resveratrol, vitamin C (ascorbic acid), dimethylethanolamine (DMAE), glycolic acid and coenzyme Q, lipoic acid, quercetin, curcumin, ascorbyl palmitate, taurine, isoflavones, lycopene, vitamin E, L-carnosine, or mixtures thereof.

24. The composition for use in a treatment of skin inflammation according to claim 15, wherein the at least one antioxidant is in a concentration of 0.01 to 0.1% by weight in relation to the composition.

25. The composition for use in a treatment of skin inflammation according to claim 15, wherein the composition is in the form of an aqueous composition, alcoholic composition, gel, cream, or lotion.
